(19) United States
(12) Patent Application Publication
(10) Pub. No.: US 2024/0342153 A1
(43) Pub. Date: Oct. 17, 2024

(54) TREATMENT OR PREVENTION OF SARS-COV-2 INFECTION USING (S)-CRIZOTINIB

(71) Applicants: Vanda Pharmaceuticals Inc., Washington, DC (US); The Board of Trustees of the University of Illinois, Urbana, IL (US)

(72) Inventors: Bellur PRABHAKAR, Oak Brook, IL (US); Prabhakaran KUMAR, Chicago, IL (US); Sandra SMIESZEK, Washington, DC (US); Bartlomiej PRZYCHODZEN, Washington, DC (US); Vuk KOPRIVICA, Bethesda, MD (US)

(21) Appl. No.: 18/294,587

(22) PCT Filed: Aug. 3, 2022
(86) PCT No.: PCT/US2022/074448

§ 371 Date: Feb. 2, 2024

Related U.S. Application Data
(60) Provisional application No. 63/203,878, filed on Aug. 3, 2021.
(60) Provisional application No. 63/264,083, filed on Nov. 15, 2021.

(51) Int. Cl. A61K 31/4545; A61P 31/14

(52) U.S. Cl. CPC A61K 31/4545; A61P 31/14

(57) ABSTRACT

Embodiments of the invention related generally to the treatment or prevention of infection by severe acute respiratory syndrome coronavirus 2 (SARS-CoV-2) and, more particularly, to the use of (S)-crizotinib or a pharmaceutically-acceptable salt thereof in such treatment or prevention. In one embodiment, the invention provides a method of treating a patient diagnosed with SARS-CoV-2, the method comprising: administering to the patient a dose of (S)-crizotinib or a pharmaceutically-acceptable salt thereof.

### CROSS-REFERENCE TO RELATED APPLICATIONS

This application claims the benefit of co-pending US Provisional Patent Application Ser. Nos. 63/203,878, filed 3 Aug. 2021, and 63/264,083, filed 15 Nov. 2021, each of which is hereby incorporated herein as though fully set forth.

### BACKGROUND

### SARS-CoV-2

Severe acute respiratory syndrome coronavirus 2 (SARS-CoV-2 or sometimes 2019-nCoV), is a spherical positive single-stranded RNA virus that is the causative agent of coronavirus disease 2019 (COVID-19). SARS-CoV-2 has infected over 126 million individuals worldwide and is the cause of over 2.7 million deaths. SARS-CoV-2 is a strain of the SARS-CoV species in the betacoronavirus genus.

As with other coronaviruses, SARS-CoV-2 has four structural proteins—the S (spike), E (envelope), M (membrane), and N (nucleocapsid). The S, E, and M proteins form the viral envelope, while the N protein, located in the core of the viral particle, binds the viral RNA and is responsible for its conformation into the viral particle. Some betacoronaviruses also include a hemagglutinin-esterase (HE) protein on the particle surface, which may enhance entry into the host cell.

The spike proteins are trimeric class I fusion proteins, heavily glycosylated, and project from the virion surface, promoting attachment to and entry into host cells. In some coronaviruses, the S protein consists of two subunits, S1 and S2, on the surface of the viral particle. In other coronaviruses, including SARS-CoV-2, the S protein includes S1 and S2 domains but remains intact on the viral particle surface until cleavage inside endocytic vesicles during viral entry.

Significant structural rearrangement of the S protein is involved in its fusion with a host cell membrane. Specifically, the receptor-binding domain (RBD) of the S1 subunit of the S protein undergoes a conformational movement from a “down” conformation, in which the binding receptors of the S1 subunit are inaccessible, to an “up” conformation, in which the receptors are accessible. The “up” conformation is believed to be less stable than the “down” conformation.

The S2 domain controls entry of the virus into the host cell. Angiotensin-converting enzyme 2 (ACE2), a type I membrane protein, is expressed widely across human tissues, including lung, heart, kidney, intestine, and adipose tissue. ACE2 is regarded as the host cell receptor for the earlier SARS-CoV strain and is a target of the SARS-CoV-2 S protein RBD. RBD binding is believed to occur on the outer surfaces of the ACE2 protein, while angiotensin substrate binding occurs within a deep cleft of the protein.

The 3.5-angstrom-resolution structure of the S protein is now know. As noted above, the S protein is cleaved into two its S1 and S2 subunits. This cleavage of S proteins by host proteases is critical for viral infection and its exit from the cell via lysosomes.

During infection, the S protein is cleaved by host cell proteases, exposing a fusion peptide of the S2 domain. Cleavage of the S protein occurs between the S1 and S2 domains and subsequently within the S2 domain (S2′) proximal to the fusion peptide. This leads to the fusion of viral and cellular membranes and the release of the viral genome into the cytoplasm of the host cell. Cleavage at both sites is believed to be necessary for viral entry into a host cell.

### Crizotinib

Crizotinib (3-[1-(2,6-dichloro-3-fluoro-phenyl)-ethoxy]-5-(1-piperidin-4-yl-1H-pyrazol-4-yl)-pyridine-2-ylamine) is a tyrosine kinase inhibitor. Its (R) enantiomer, marketed as XALKORI®, is approved in the United States for the treatment of metastatic non-small cell lung cancer (NSCLC) with anaplastic lymphoma kinase (ALK)- or ROS proto-oncogene 1 (ROS1)-positive tumors and for pediatric patients with relapsed or refractory systemic anaplastic large cell lymphoma (ALCL) that is ALK-positive.

Approved doses of (R)-crizotinib are 250 mg twice daily, delivered orally, for metastatic NSCLC and 280 mg/m² of body surface, twice daily, delivered orally, for systemic ALCL.

(S)-crizotinib has the structure of Formula I below.

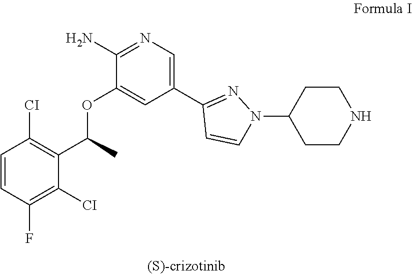

(R)-crizotinib has the structure of formula II below.

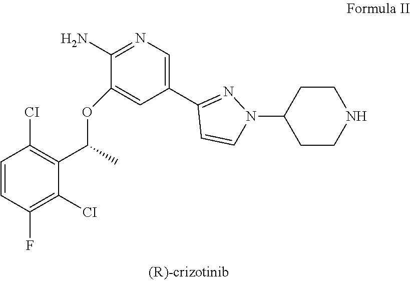

The (R) and(S) enantiomers of crizotinib are known to have very different affinities for kinases. Huber et al., for example, report a 20-fold greater MuT Homolog 1 (MTH1; Nudix hydrolase 1, NUDT1) inhibitory potency in(S)-crizotinib as compared to (R)-crizotinib. More specifically, while the approved (R) enantiomer binds with high affinity to more than 20 kinases and efficiently inhibits at least 10, the(S) enantiomer binds with high affinity only to the MTH1 kinase, and no proteins are significantly bound by both enantiomers. As such, at least with respect to kinase inhibition, the enantiomers may have distinct molecular mechanisms of action.

MTH1, a Nudix pyrophosphatase, is a nucleotide pool sanitizing enzyme. It has been investigated as a therapeutic target in RAS-driven cancers because elevated expression appears to facilitate RAS-driven transformation. Some researchers have hypothesized that, during replication, MTH1 promotes malignancy in oncogenic RAS-harboring cells by preventing the incorporation of nucleic bases damaged by the oxidative stress in which cancer cells operate. By inhibiting MTH1 expression, oxidized bases are incorporated into the DNA or RNA, leading to inefficient replication due to the high rate of error correction and subsequent cell death. This has made inhibition of MTH1 rather than inhibition of RAS itself a potentially viable cancer treatment strategy.

The results for all MTH1 inhibitors, including(S)-crizotinib, have been mixed, however. Some MTH1 inhibitors have been found to have little or no anticancer activity. Others cause cancer cell death, but do so independent of MTH1 inhibition. For example, studies have shown that CRISPR or siRNA silencing of the MTH1 gene does not affect cancer cell viability but that MTH1 inhibitors nevertheless kill these same cells. Thus, while(S)-crizotinib may prove useful in cancer treatment, it may do so independent of its ability to inhibit MTH1.

### SUMMARY OF THE INVENTION

Embodiments of the invention relate generally to the treatment or prevention of infection by severe acute respiratory syndrome coronavirus 2 (SARS-CoV-2) and, more particularly, to the use of (S)-crizotinib or a pharmaceutically-acceptable salt thereof in such treatment or prevention.

In one embodiment, the invention provides a method of treating infection by severe acute respiratory syndrome coronavirus 2 (SARS-CoV2) in an individual, the method comprising: administering to the individual a dose of (S)-crizotinib or a pharmaceutically-acceptable salt thereof effective to treat COVID-19.

In another embodiment, the invention provides a method of preventing infection by severe acute respiratory syndrome coronavirus 2 (SARS-CoV2) in an individual at risk of such infection, the method comprising: administering to the individual a dose of (S)-crizotinib or a pharmaceutically-acceptable salt thereof effective to prevent the infection with SARS-CoV2.

In another embodiment, the invention provides a method of preventing symptomatic infection by severe acute respiratory syndrome coronavirus 2 (SARS-CoV2) in an individual at risk of such infection, the method comprising: administering to the individual a dose of (S)-crizotinib or a pharmaceutically-acceptable salt thereof effective to prevent the manifestation of symptoms of a COVID-19 infection.

In still another embodiment, the invention provides a pharmaceutical composition comprising: (S)-crizotinib or a pharmaceutically-acceptable salt thereof; and a pharmaceutically-acceptable excipient or carrier.

In yet another embodiment, the invention provides(S)-crizotinib or a pharmaceutically-acceptable salt thereof for use in the treatment or prevention of severe acute respiratory syndrome coronavirus 2 (SARS-CoV2) infection in an individual, the treatment or prevention comprising: administering to the individual an amount of (S)-crizotinib or a pharmaceutically-acceptable salt thereof effective to treat or prevent a COVID-19 infection.

In another embodiment, the invention provides the use of (S)-crizotinib or a pharmaceutically-acceptable salt thereof in the manufacture of a medicament for the treatment or prevention of severe acute respiratory syndrome coronavirus 2 (SARS-CoV2) infection in an individual.

In still yet another embodiment, the invention provides a method of treating a viral infection in a patient, the method comprising: inhibiting viral nucleic acid replication in the patient by administering to the patient a dose of (S)-crizotinib or a pharmaceutically-acceptable salt thereof sufficient to inhibit MTH1 expression in the patient.

### BRIEF DESCRIPTION OF THE DRAWINGS

These and other features of this invention are more readily understood from the following detailed description of the various aspects of the invention taken in conjunction with the accompanying drawings that depict various embodiments of the invention, in which:

FIG. 1 shows data related to the comparative levels of pseudovirus entry inhibition by (S)-crizotinib and (R)-crizotinib;

FIG. 2 shows data related to the comparative efficiency of pseudovirus entry inhibition by(S)-crizotinib and (R)-crizotinib;

FIG. 3 shows data related to the comparative inhibition of SARS-CoV-2-induced cytopathic effects by(S)-crizotinib and (R)-crizotinib across three cell lines; and

FIG. 4 shows comparative drug toxicities of (S)-crizotinib and (R)-crizotinib across three cell lines.

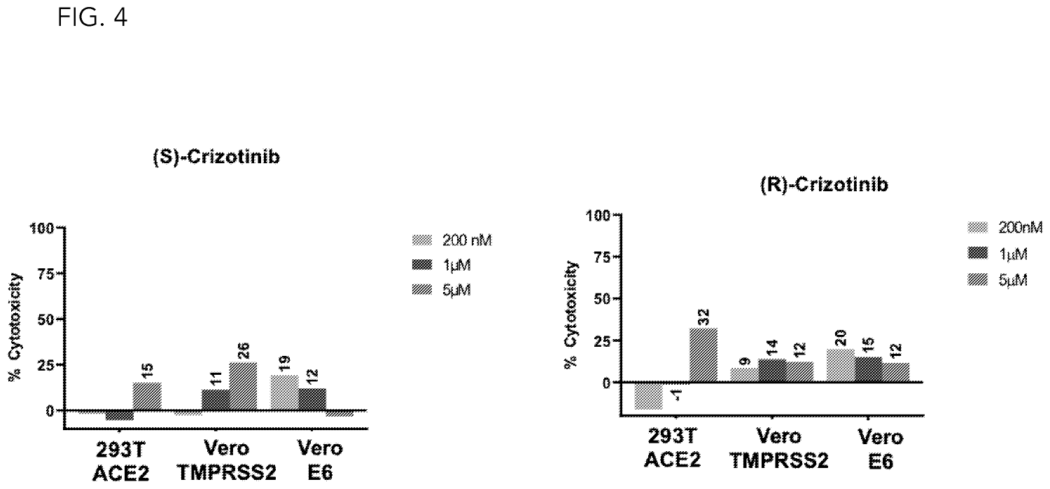

It is noted that the drawings of the invention are not to scale. The drawings are intended to depict only typical aspects of the invention, and therefore should not be considered as limiting the scope of the invention. In the drawings, like numbering represents like elements among the drawings.

### DETAILED DESCRIPTION

The present invention relates to the discovery that crizotinib is effective in preventing infection by SARS-CoV-2, and, surprisingly, that(S)-crizotinib is significantly more effective than (R)-crizotinib in doing so. This may be attributable to(S)-crizotinib's ability to inhibit MTH1. During viral replication, inhibition of MTH1 results in the incorporation of oxidatively damaged nucleotide bases, resulting in high error correction and less efficient RNA synthesis.

In accordance with the present invention, SARS-CoV-2 pseudotyped virus particles are produced by transfecting HEK293T cells with the following plasmids: (1) lentiviral backbone plasmid-pHAGE-CMV-Luc2-IRES-ZsGreen, which expresses luciferase and ZsGreen reporters, (2) PHDM-Hgpm2-lentiviral helper plasmid, which expresses HIV Gag-Pol, (3) pHDM-tat1b, a lentiviral helper plasmid expressing HIV Tat, and (4) pRC-CMV-Rev1b, a lentiviral helper plasmid expressing HIV Rev. These constituted bald pseudovirus particles.

Additional pseudotyped virus particles are produced by transfecting HEK293T cells with a fifth plasmid, pHDM, expressing the SARS-CoV-2 Wuhan-Hu-1 Spike envelope glycoprotein.

Human ACE2-expressing 293T cells (25×10³) are seeded in a 96-well plate and infected with the bald pseudovirus particles only (negative control) or the spike glycoprotein-expressing pseudovirus particles using 50 μL of a 2% fetal bovine serum (FBS) infection medium, 150 μL of pseudovirus-containing 10% Dulbecco's Modified Eagle Medium (DMEM) medium, and 1 μM (S)-crizotinib or (R)-crizotinib. After 72 hours, the cells are lysed and the luciferase activity is determined by luciferase assay, wherein the spent medium is removed and 60 μL of cell lysis buffer reagent added to each well, the plates are incubated for 15 minutes with shaking, 40 μL luciferin substrate added, and luminescence read with one minute integration and delay time.

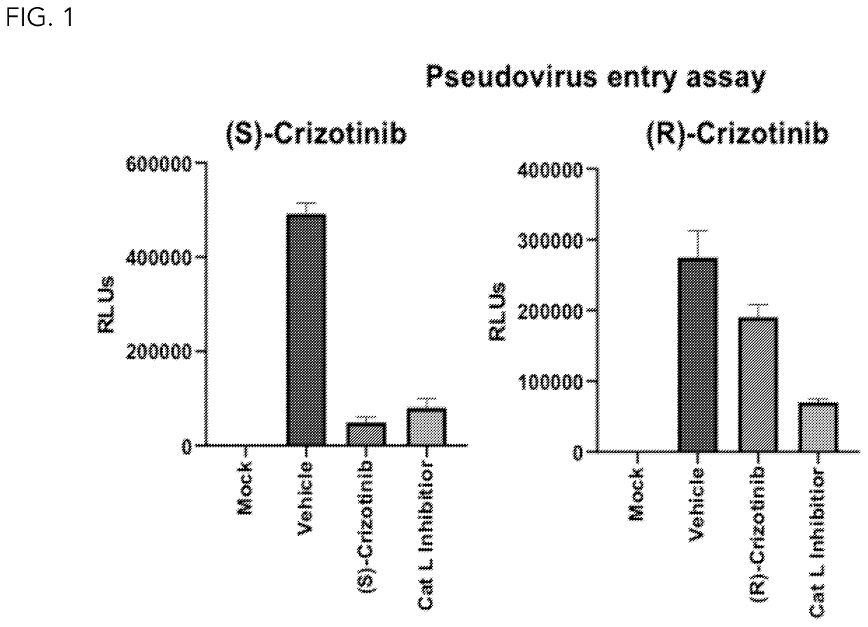

These tests are performed three times and the averaged results are shown in FIG. 1. In panel A, it can be seen that no pseudoviral entry is noted in the negative control cells (labeled as “Mock,” i.e., those not infected with the SARS-CoV-2 spike glycoprotein or treated with inhibitor). The positive control cells (labeled as “Vehicle,” i.e., those infected with the SARS-CoV-2 spike glycoprotein but not treated with inhibitor), on the other hand, exhibited pseudoviral entry used as the baseline for comparison to those cells treated with(S)-crizotinib or a cathepsin L (CatL) inhibitor. CatL is a lysosomal cystein protease known to be involved in viral entry by cleaving the S1/S2 cleavage site of severe acute respiratory syndrome coronavirus (SARS-CoV) and believed to be similarly involved in cleaving the S1/S2 cleavage site of SARS-CoV-2. Inhibition of CatL is shown to be effective in suppressing SARS-CoV infection. As can be seen in panel A of FIG. 1, (S)-crizotinib is highly effective in inhibiting pseudovirus entry and more effective than is the CatL inhibitor.

Panel B of FIG. 1 shows similar data with respect to (R)-crizotinib. However, while (R)-crizotinib inhibits pseudoviral entry, it is not as effective in doing so as either(S)-crizotinib or the CatL inhibitor.

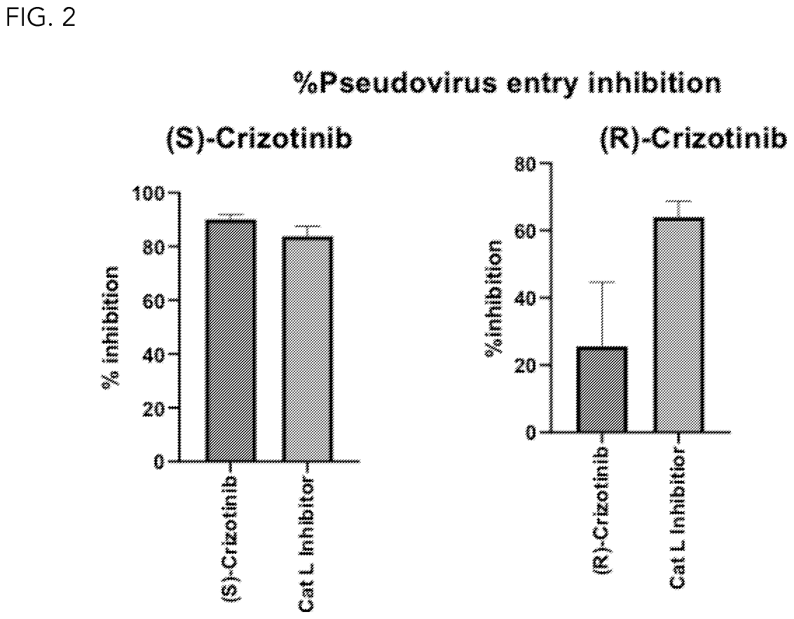

FIG. 2 shows the inhibition of pseudovirus entry as a percentage, compared to the CatL inhibitor, for both(S)-crizotinib (panel A) and (R)-crizotinib (panel B). The percentage is calculated according to the following formula.

$$\% = 100 \times [ 1 - ( X - MIN ) / ( MAX - Min ) ] ,$$

where X is the RLU of the crizotinib-treated wells of FIG. 1, MIN is the RLU of the Mock or negative control wells of FIG. 1, and MAX is the RLU of the Vehicle or positive control wells of FIG. 1.

As can be seen in panel A of FIG. 2, (S)-crizotinib is approximately 90% effective in inhibiting pseudoviral entry, as compared to approximately 80% efficiency for the CatL inhibitor. In comparison, as shown in panel B of FIG. 2, (R)-crizotinib is approximately 25% effective in inhibiting pseudoviral entry, as compared to approximately 60% efficiency for the CatL inhibitor.

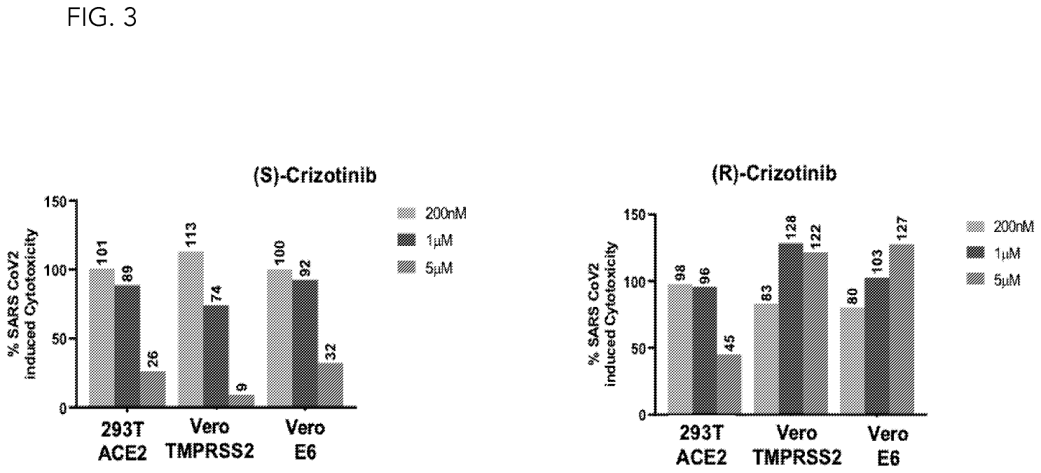

FIG. 3 shows data related to the comparative inhibition of SARS-CoV-2-induced cytopathic effects by(S)-crizotinib (panel A) and (R)-crizotinib (panel B) at varying doses across three cell lines—the ACE2-expressing 293T cells described above and for which data are shown in in FIGS. 1 and 2, Vero E6 cells expressing transmembrane protease, serine 2 (TMPRSS2), and Vero E6 cells. TMPRSS2 is believed to cleave the S2 and S2′ domains described above and inhibition of TMPRSS2 has been shown to suppress SARS-CoV infection.

The Vero E6 and Vero E6-TMPRSS2 cells are prepared similarly to the 293T-ACE2 cells described above, although here, all three cell lines are infected with SARS-CoV-2, Isolate USA-WA1/2020 obtained from BEI Resources. Percentage inhibition is calculated as described above.

As can be seen in FIG. 3, (S)-crizotinib inhibits SARS-CoV-2-induced cytotoxic effects in a concentration-dependent manner in all three cell lines. At the highest concentration of 5 μM, the cytotoxic effects of SARS-CoV-2 are inhibited by 74%, 91%, and 68%, respectively, in the 293T-ACES, Vero E6-TMPRSS2, and Vero E6 cell lines.

The results for (R)-crizotinib, shown in panel B of FIG. 3, are neither as significant nor as consistent. In the 293T-ACES cell line, inhibition increases with the concentration of (R)-crizotinib. However, even at 5 μM, where inhibition is greatest at 55%, (R)-crizotinib is markedly less effective than(S)-crizotinib. Inhibition in the Vero E6-TMPRSS2 and Vero E6 cell lines is not concentration-dependent, with the highest concentration (5 μM) showing lower inhibition than the lowest concentration (200 nM) and greater SARS-CoV-2-induced cytotoxic effects than in untreated cells.

These results provide support for the use(S)-crizotinib or a pharmaceutically-acceptable salt thereof in the treatment or prevention of SARS-CoV-2 infection. For example, (S)-crizotinib or a pharmaceutically-acceptable salt thereof may be administered to an individual infected with SARS-CoV-2 or at risk for such infection in an amount sufficient to decrease SARS-CoV-2-induced effects and/or further viral entry in an infected individual or to prevent viral entry and the onset of such effects in an individual at risk of infection.

In one aspect of the present invention, the(S) enantiomer or a pharmaceutically-acceptable salt thereof is used at the same or similar doses as those approved for the (R) enantiomer in the treatment of NSCLC and systemic ALCL—250 mg or 280 mg/m² body surface area, twice daily. (R)-crizotinib is currently available in 200 mg and 250 mg capsules.

Pharmaceutical compositions comprising(S)-crizotinib or a pharmaceutically-acceptable salt thereof may similarly be provided in capsule form and in similar dosages. For example, (S)-crizotinib or a pharmaceutically-acceptable salt thereof may be orally administered at a dose of 250 mg/m² or 280 mg/m² body surface area twice daily. As will be apparent to one skilled in the art, however, other dosage forms with other amounts of (S)-crizotinib or a pharmaceutically-acceptable salt thereof are also possible and within the scope of the invention. The particular dosage form and dose will be determined based on a number of considerations, including, for example, the patient's age, weight, and overall health, the severity of infection to be treated, any medicaments which the patient may be co-administered, and any adverse reactions to which the patient may be susceptible.

For example, (R)-crizotinib has been associated with QT interval prolongation, bradycardia, vision loss, and hepatotoxicity. It is also contraindicated for patients with interstitial lung disease (ILD)/pneumonitis. Patients receiving(S)-crizotinib may similarly be monitored for these conditions.

Patients administered (R)-crizotinib are also advised to avoid concomitant use of strong CYP3A inhibitors, strong CYP3A inducers, and CYP3A substrates. Where concomitant use with strong CYP3A inhibitors is unavoidable, patients are advised to reduce the dose of (R)-crizotinib. Where concomitant use with CYP3A substrates is unavoidable, patients are advised to reduce the dose of the CYP3A substrate. Patients receiving(S)-crizotinib or a pharmaceutically-acceptable salt thereof may similarly be advised as to such concomitant uses.

(R)-crizotinib can cause fetal harm and is not recommended for use in patients who are pregnant or who are breastfeeding. Patients receiving(S)-crizotinib may be similarly advised.

Reduced doses of (R)-crizotinib are advised for patients with moderate or severe hepatic impairment or severe renal impairment. Patients receiving(S)-crizotinib may be similarly advised.

Pharmaceutical compositions comprising(S)-crizotinib or a pharmaceutically-acceptable salt thereof and pharmaceutically-acceptable carriers and/or excipients are within the scope of the invention, including compositions comprising(S)-crizotinib or a pharmaceutically-acceptable salt thereof as the sole or primary active ingredient.

Formulation methods applicable to(S)-crizotinib or pharmaceutically-acceptable salts thereof will vary, of course, depending on the desired dosage form. Relevant factors in preparing such a formulation include particle size, polymorphism, pH, solubility, stability, etc., as one skilled in the art will appreciate. Formulations may be suitable for enteral administration (e.g., tablets, capsules, liquid suspension) or parenteral administration (e.g., liquid, lyophilized), and formulated for immediate, sustained, delayed, or pulsatile release.

Suitable dosage forms include capsules together with one or more inactive ingredient, such as colloidal silicon dioxide, microcrystalline cellulose, anhydrous dibasic calcium phosphate, sodium starch glycolate, magnesium stearate, and gelatin capsule shells.

Base addition salts of (S)-crizotinib may be formed using known techniques, as will be appreciated by one skilled in the art. The particular salt formulation employed may vary based, for example, on the intended dosage form, route of administration, desired solubility and dissolution rate, intended release profile, compatibility with excipients, etc. Suitable salt formulations of (S)-crizotinib may include, for example, hydrochloride, mesylate, hydrobromide, acetate, and fumarate, though other pharmaceutically-acceptable salt forms are possible.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method of treating or preventing a patient diagnosed with severe acute respiratory syndrome coronavirus 2 (SARS-CoV-2), the method comprising:
administering to the patient a dose of (S)-crizotinib or a pharmaceutically-acceptable salt thereof.

2. The method of claim 1, wherein the dose is 250 mg twice per day.

3. The method of claim 1, wherein the dose is 280 mg/m² twice per day based on body surface area.

4. The method of claim 1, wherein administering includes orally administering the dose of (S)-crizotinib or a pharmaceutically-acceptable salt thereof.

5-8. (canceled)

9. A pharmaceutical composition comprising:
(S)-crizotinib or a pharmaceutically-acceptable salt thereof; and
a pharmaceutically-acceptable excipient or carrier.

10. The pharmaceutical composition of claim 9, wherein the(S)-crizotinib or a pharmaceutically-acceptable salt thereof is the primary or sole active ingredient.

11. The pharmaceutical composition of claim 9 in the form of a capsule.

12. The pharmaceutical composition of claim 9, wherein the(S)-crizotinib or a pharmaceutically-acceptable salt thereof is present in a dose effective to treat or prevent infection by severe acute respiratory syndrome coronavirus 2 (SARS-CoV-2).

13. The pharmaceutical composition of claim 9, comprising about 200 mg of (S)-crizotinib or a pharmaceutically-acceptable salt thereof.

14. The pharmaceutical composition of claim 9, comprising about 250 mg of (S)-crizotinib or a pharmaceutically-acceptable salt thereof.

15-23. (canceled)

24. A method of treating or preventing a viral infection in a patient, the method comprising:
inhibiting viral nucleic acid replication in the patient by administering to the patient a dose of (S)-crizotinib or a pharmaceutically-acceptable salt thereof sufficient to inhibit MTH1 expression in the patient.

25. The method of claim 24, wherein the viral infection includes infection by severe acute respiratory syndrome coronavirus 2 (SARS-CoV-2).

26. The method of claim 24, wherein the dose is 250 mg twice per day.

27. The method of claim 24, wherein the dose is 280 mg/m² twice per day based on body surface area.

28. The method of claim 24, wherein administering includes orally administering the dose of (S)-crizotinib or a pharmaceutically-acceptable salt thereof.
